(19) United States
(12) Patent Application Publication
(10) Pub. No.: US 2020/0102618 A1
(43) Pub. Date: Apr. 2, 2020

(54) LIQUID BIOPSY FOR cfRNA

(71) Applicant: NANTOMICS, LLC, Culver City, CA (US)

(72) Inventors: Kathleen DANENBERG, Culver City, CA (US); Shahrooz RABIZADEH, Culver City, CA (US); Joshua USHER, Culver City, CA (US); Yolanda JAIMES, Culver City, CA (US)

(21) Appl. No.: 16/494,683

(22) PCT Filed: Mar. 15, 2018
(86) PCT No.: PCT/US2018/022747

§ 371 Date: Sep. 16, 2019

Related U.S. Application Data
(60) Provisional application No. 62/473,273, filed on Mar. 17, 2017.
(60) Provisional application No. 62/522,509, filed on Jun. 20, 2017.
(60) Provisional application No. 62/593,534, filed on Dec. 1, 2017.

(51) Int. Cl. C12Q 1/6886

(52) U.S. Cl. CPC C12Q 1/6886; C12Q 2600/106; C12Q 2600/158; C12Q 2600/118

(57) ABSTRACT

cfRNA is used to identify and quantitate expression levels of disease related genes and further allows for non-invasive monitoring of changes in such genes. Moreover, quantitative analysis of disease related genes will enable prediction of treatment response where the treatment is dependent on the presence of the disease related gene.

This application claims priority to our copending US provisional applications having the Ser. No. 62/473,273, filed Mar. 17, 2017, 62/522,509, filed Jun. 20, 2017, and 62/593,534, filed Dec. 1, 2017.

### FIELD OF THE INVENTION

The field of the invention is systems and methods of detection and quantification of circulating free RNA (cfRNA), especially as it relates to cfRNA from tumor cells.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Over the last decade, cancer therapy has changed from a general chemotherapy based therapy in combination with surgery and radiation to a more personalized treatment that takes into account the genetic variability of tumors across patients. Therefore, treatment plans often now require identification of molecular markers that allow a more targeted therapy. In many cases, such information is obtained by analysis of various nucleic acid molecules from cancer tissue biopsies. However, tissue biopsies are often limited to initial diagnosis or surgery, and later biopsies tend to incur significant risk and discomfort to the patient. Moreover, tumor tissue biopsies tend to be problematic in terms of sampling bias and limited ability to monitor nucleic acid molecules as tumor markers in patients during the course of therapy.

While it was known that nucleic acid molecules from tumor and non-tumor cells can be obtained from blood (see e.g., Clin Canc. Res. (1999) Vol 5, 1961-1965; Canc Res. (1977) 37:646-650), it was not clear whether or not these nucleic acids were associated or bound with any carrier or other structure. Indeed, more recently it was discovered that RNA can originate from various sources, including circulating tumor cells (see e.g., WO 2017/180499), exosomes (see e.g., WO 2015/082372), and carrier proteins (see e.g., WO 2010/079118, or Proc. Natl. Acad. Sci. (1985) 82, 3455).

Unfortunately, and possibly due to the different locations/associations of RNA with various carriers or other structures, accurate quantification of circulating nucleic acids has often been problematic. For example, disease status detection in neuroblastoma using cell free RNA was shown not to be a reliable alternative to whole cell RNA analysis (see e.g., Pediatr Blood Cancer. 2010 Jul. 1; 54(7):897-903). Similarly, while being able to detect relatively small quantities of cfRNA from mutated or improperly fused genes in the blood regardless of their particular association as described in WO 2016/077709, the detected quantities of such RNAs varied significantly. Moreover, it remained unknown whether any of the detected quantities was a reflection of physiological reality within a cell or a function of stability of the particular RNA in question. For example, data in the '709 publication indicate that the quantities of cfRNA encoding PD-1/PD-L1 is often highly variable and may depend on the sample, patient condition, and other factors. Consequently, there has to date been no report of using PD-L1 cfRNA expression levels as a prognostic agent and/or indicator to determine eligibility of cancer patients for anti-PD-1/PD-L1 therapy.

Therefore, even though numerous methods of nucleic acid analysis from biological fluids are known in the art, all or almost all of them suffer from various disadvantages. Thus, there remains a need for improved systems and methods for cfRNA analysis.

### SUMMARY OF THE INVENTION

The inventive subject matter is directed to various compositions and methods of using cfRNA levels of one or more cfRNA to predict treatment response, to track treatment, and/or to diagnose a cancer. In especially preferred aspects, the inventors discovered that expression threshold levels for certain cfRNA, and especially PD-L1 and HER2, can be determined that are predictive for treatment response for certain cancers.

In one aspect of the inventive subject matter, method of predicting treatment response of an individual with cancer to treatment with a checkpoint inhibitor that includes a step of obtaining blood from the individual and isolating cfRNA from the blood, wherein the cfRNA encodes a checkpoint inhibition gene and a further step of quantifying the cfRNA using quantitative PCR method. A positive treatment response is then predicted when the quantity of the cfRNA is above a threshold level.

In preferred embodiments, the checkpoint inhibitor is an antibody against PD1 or PD-L1 and the cfRNA is PD-L1 cfRNA. Moreover, it is generally preferred that the step of isolating the cfRNA uses at least one of RNA stabilization and cell preservation. Most typically, the quantitative PCR method includes real time PCR, preferably with β-actin as an internal standard. Where PD-L1 is quantified, the threshold level may be ΔΔCT>10 for PD-L1 relative to β-actin. Additionally, where desired, at least one second cfRNA may be quantified using the quantitative PCR method. While not limiting to the inventive subject matter, contemplated second cfRNAs may encode TIM3 or LAG3, a gene having a tumor and patient specific mutation, a tumor associated gene, or a cancer specific gene.

In another aspect of the inventive subject matter, the inventors also contemplate a method of monitoring treatment of an individual with cancer that includes a step of obtaining blood from the individual and isolating cfRNA from the blood, wherein the cfRNA encodes a checkpoint inhibition gene, or wherein the cfRNA encodes a tumor associated or cancer specific gene, or wherein the cfRNA encodes a gene having a tumor and patient specific mutation; a step of quantifying the cfRNA using quantitative PCR method; and a step of updating a patient record using the quantity of the cfRNA.

For example, suitable checkpoint inhibition gene include PD-L1, TIM3, or LAG3, tumor associated or cancer specific gene include CEA, MUC1, brachyury, HER2, PCA3, or AR-V7, and suitable genes having a tumor and patient specific mutation preferably encode a neoepitope. As noted above, it is generally preferred that the step of isolating the cfRNA uses RNA stabilization and cell preservation, and that the quantitative PCR method includes real time PCR (e.g., using β-actin as an internal standard). The patient record may be updated when the quantity of the cfRNA is ΔΔCT>5 for HER2 relative to β-actin or ΔΔCT>10 for PCA3 relative to β-actin.

In still another aspect of the inventive subject matter, the inventors contemplate a method of detecting prostate cancer that includes a step of obtaining blood from the individual and isolating cfRNA from the blood, wherein the cfRNA encodes PCA3 or a splice variant 7 of an androgen receptor; a further step of quantifying the cfRNA using quantitative PCR method; and a still further step of diagnosing the individual as having cancer when the cfRNA quantity is above a threshold level. Most typically, the individual is diagnosed as having cancer when the quantity of PCA3 cfRNA is ΔΔCT>10 relative to β-actin.

Where desired, at least a second cfRNA may be quantified that encodes a gene having a tumor and patient specific mutation, a tumor associated gene, a cancer specific gene, or a checkpoint inhibition gene. Therefore, such second genes include PD-L1, LAG3, TIM3, AR-V7, PSA, and PSMA.

In yet another aspect of the inventive subject matter, the inventors also contemplate a method of treating a cancer that includes the steps of administering a drug to an individual diagnosed with a PD-L1 negative cancer; monitoring treatment of the individual by isolating cfRNA from the blood, wherein the cfRNA encodes PD-L1; quantifying the cfRNA using quantitative PCR method; and including a checkpoint inhibitor to the treatment upon detection of the cfRNA.

In such methods, it is typically contemplated that the PD-L1 negative cancer is a solid cancer (e.g., breast cancer), and/or that the drug is afinitor. Most typically, the step of quantifying cfRNA uses real-time PCR, and the checkpoint inhibitor is included when the cfRNA is detected and increases over time. In further preferred aspects of such methods, the checkpoint inhibitor is included when the cfRNA is detected and the cfRNA level is ΔΔCT>10 relative to β-actin.

Moreover, the inventors also contemplate a method of determining an immune signature in a patient that includes a step of determining quantities of distinct cfRNA molecules in blood of an individual, wherein the cfRNA molecules encode distinct checkpoint inhibition genes (e.g., PD-L1, TIM3, LAG3). Typically, the step of determining is performed prior to or during treatment with at least one of a checkpoint inhibitor, a chemotherapeutic drug, an immune therapeutic drug, and radiation treatment.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 depicts graphs comparing plasma concentrations for cfDNA and cfRNA for healthy subjects and subjects diagnosed with cancer.

FIG. 2A depicts a graph comparing plasma concentrations for PD-L1 cfRNA for across various cancer types.

FIG. 2B depicts a graph showing plasma concentrations for PD-L1 cfRNA for healthy subjects.

FIG. 2C depicts a graph showing the linear range for plasma concentrations for PD-L1 cfRNA.

FIG. 3A depicts a graph showing the relative expression of PD-L1 cfRNA for lung cancer patients in a clinical trial.

FIG. 3B depicts data showing PD-L1 expression as measured by IHC for the lung cancer patients in the clinical trial.

FIG. 4 depicts a graph showing PD-L1 cfRNA levels for a non-responder and a responder to nivolumab and corresponding IHC staining of lung tumor samples, along with PD-L1 cfRNA levels during treatment.

FIG. 5A depicts a graph correlating PD-L1 cfRNA levels with the PD-L1 status as determined by PD-L1 IHC.

FIG. 5B depicts a graph correlating PD-L1 cfRNA levels with nivolumab response status demonstrating a clinically relevant expression threshold for PD-L1 cfRNA levels.

FIGS. 6A-6D depicts graphs comparing plasma concentrations for PD-L1 cfRNA levels of subjects diagnosed with cancer and undergoing treatment.

FIG. 7 depicts a graph illustrating PD-L1 cfRNA levels as a function of treatment with afinitor suggesting treatment with anti-PD1/PD-L1 compositions.

FIG. 8 depicts a graph correlating cancer treatment response status with overall cfRNA/beta-actin cfRNA.

FIG. 9A depicts a graph showing relative co-expression of PD-L1 and HER2 as measured by cfRNA levels.

FIG. 9B depicts a graph correlating HER2 cfRNA levels with the HER2 status as determined by HER2 IHC/FISH demonstrating a clinically relevant expression threshold for HER2 cfRNA levels.

FIG. 10 depicts a graph showing relative co-expression of PD-L1 and HER2 in gastric cancer as measured by cfRNA levels.

FIG. 11 depicts a graph correlating pertuzumab/trustuzumab treatment response with HER2 cfRNA levels.

FIG. 12 depicts cfRNA signatures for selected checkpoint relevant genes.

FIG. 13 depicts exemplary results for AR-V7 cfRNA levels and AR cfRNA levels in prostate cancer patients indicating that AR-V7 cfRNA is a suitable marker.

FIG. 14 depicts exemplary results for PCA3 cfRNA levels in non-prostate cancer and prostate cancer patients indicating that PCA3 cfRNA is a suitable marker.

### DETAILED DESCRIPTION

The inventors have discovered that cfRNA can be employed as a sensitive, selective, and quantitative marker for diagnosis, monitoring of treatment, and even as discovery tool that allows repeated and non-invasive sampling of a patient. In most preferred aspects, the cfRNA is isolated from whole blood that is processed under conditions that preserve cellular integrity and stabilize cfRNA and/or ctDNA. Notably, the ratio of cfRNA to RNA released from non-tumor cells damaged during whole blood processing under such cell preserving conditions is sufficiently high to perform quantitative analysis that can provide clinically meaningful results. Once separated from the non-nucleic acid components, the circulating nucleic acids are then quantified, preferably using real time quantitative PCR. Therefore, the inventors also contemplated kits, reagents, and instructions for isolation, monitoring, and quantification of cfRNA in blood, and especially oligonucleotides for primers suitable to quantitatively determine presence of cfRNA for specific genes as is further discussed in more detail below.

Of course, and as is discussed in more detail below, it should be appreciated that one or more desired nucleic acids may be selected for a particular disease, disease stage, specific mutation, or even on the basis of personal mutational profiles or presence of expressed neoepitopes. Alternatively, where discovery or scanning for new mutations or changes in expression of a particular gene is desired, real time quantitative PCR may be replaced or supplemented by RNAseq to so cover at least part of a patient cfRNA transcriptome. Moreover, it should be appreciated that analysis can be performed static, or over a time course with repeated sampling to obtain a dynamic picture without the need for biopsy of the tumor or a metastasis.

Viewed form a different perspective, the inventors have generally discovered various methods and compositions for blood-based RNA expression testing of circulating tumor RNA (cfRNA) that identifies and quantitates expression, and that allows for non-invasive monitoring of changes in indicators and/or drivers of disease that have heretofore only been available by protein-based analysis of biopsied tissue. For example, contemplated systems and methods allow monitoring changes in indicators and/or drivers of a disease, and/or identification of changes in drug targets that may be associated with emerging resistance to chemotherapies. Advantageously, contemplated systems and methods integrate with other omics analysis platforms, and especially GPS Cancer (that provides whole genome or exome sequencing, RNA sequence and expression analysis, and quantitative protein analysis) to establish a powerful primary analysis/monitoring combination tool in which alterations identified by an omics platform are non-invasively, molecularly monitored by systems and methods presented herein.

In some embodiments, the inventors contemplate method of determining status of a (e.g., solid) cancer in a patient that includes a step of selecting a cancer related gene on the basis of at least one of a known association of a gene with the cancer and/or a prior omics analysis of cancer tissue in the patient. In another step, cfRNA of the cancer related gene is quantified in a bodily fluid (e.g., whole blood, serum, or plasma) of the patient, and in a further step the quantity of the cfRNA is associated with the cancer status. Alternatively, or in addition to the cancer related gene, other cfRNA may also be monitored. For example, the cancer status may be susceptibility of the cancer to treatment with a drug, or presence or absence of the cancer in the patient. Most typically, the cancer related gene is a cancer associated gene, a cancer specific gene, or a gene encoding a patient and tumor specific neoepitope (which may be determined using GPS cancer omics analysis). In further contemplated aspects, as described in more detail below, the step of quantifying will include isolation of the cfRNA under RNA stabilization and cell preservation, and/or the step of quantifying includes real time quantitative PCR of a cDNA prepared from cfRNA.

In other embodiments, the inventors also contemplate methods of selecting a patient for treatment with a checkpoint inhibitor that may include a step of obtaining a bodily fluid from the patient and quantifying a cfRNA in the bodily fluid for at least one checkpoint inhibition related gene. Among other suitable cfRNAs, especially contemplated cfRNA include those encoding PD-L1 and HER2. Of course, it should be recognized that the cfRNA need not encode the full gene, but may be a fragment of the gene under investigation. The quantity of the cfRNA is then compared against a threshold value that associates the quantity with a likely treatment outcome. Consequently, and among other options, treatment outcomes may be related to treatments with one or more checkpoint inhibitors (e.g., antibody or antibody fragment against PD-1, PD-L1, TIM3, and/or LAG3) and/or treatment with antibodies targeting various receptors (e.g., EGFR, ERCC1, IGF1, HER2, etc.)

Therefore, the inventors also contemplate various methods of treating a cancer that includes a step of determining cfRNA quantities of a first and a second marker in a blood sample of a patient, wherein the first marker is a checkpoint inhibition related gene, and wherein the second marker is one of a cancer associated gene, a cancer specific gene, or a gene encoding a patient and tumor specific neoepitope. It is further contemplated that the quantities of the first and second markers in such methods are (e.g., positively) associated. The quantity of the second marker may then be used to determine treatment with a checkpoint inhibitor. For example, the first marker is PD-1 or PD-L1 (or other checkpoint inhibition related marker), and the second marker is HER2. Likewise, the first marker is PD-1 or PD-L1 (or other checkpoint inhibition related marker), and the second marker is a cfRNA encoding a neoepitope.

In still other embodiments, the inventors also contemplate a method of determining an immune signature in a patient that includes a step of determining cfRNA quantities of a plurality of markers in a blood sample of the patient, wherein the plurality of markers comprise checkpoint inhibition related genes. Most typically, the step of determining is performed prior to or during treatment with at least one of a checkpoint inhibitor, a chemotherapeutic drug, an immune therapeutic drug, and radiation treatment. Moreover, contemplated methods may further comprise a step of determining a cfRNA quantity of at least one costimulatory marker, and/or a step of generating or updating a treatment plan based on the determined quantities.

In general, it is contemplated that cfRNA analysis is performed using any bodily fluid that contains cfRNA. Therefore, suitable bodily fluids include whole blood, plasma, serum, lymphatic fluid saliva, ascites fluid, spinal fluid, urine, etc., each of which may be fresh or preserved/frozen. However, it is especially preferred that the cfRNA analysis uses whole blood as a biological sample. Whole blood is readily obtained without significant patient discomfort and can be processed in a simple and effective manner As is further described in more detail below, the inventors discovered that the protocols used for removal of cells from whole blood had a significant impact on stability and yield of the RNA. Notably, the inventors discovered that quantitative cfRNA analyses were significantly improved where the cells were removed from the whole blood under conditions that maintained integrity of the cells. While not wishing to be bound by any theory or hypothesis, the inventors contemplate that cell lysis of non-tumor cells in blood is a substantial contributing factor in release of non-cfRNA. Moreover, certain RNA stabilizing agents may also adversely affect white and red blood cells, and as such contribute to release of non-cfRNA into the plasma.

For example, for the analyses presented herein, specimens were accepted as 10 ml of whole blood drawn into cell-free RNA BCT® tubes or cell-free DNA BCT® tubes (which are both commercially available from Streck Inc.,7002 S. 109^{th} St., La Vista Nebr. 68128) containing RNA or DNA stabilizers, respectively. Advantageously, cfRNA is stable in whole blood in the cell-free RNA BCT tubes for seven days while ctDNA is stable in whole blood in the cell-free DNA BCT Tubes for fourteen days, allowing time for shipping of patient samples from various locations without the degradation of cfRNA or ctDNA. However, it should be noted that numerous alternative collection tubes and compositions are also deemed suitable so long as the RNA stabilization agents will not lead to substantial cell lysis (e.g., equal or less than 3%, equal or less than 1%, or equal or less than 0.1%, or equal or less than 0.01%, or equal or less than 0.001%) lyse white and/or red blood cells. Viewed from a different perspective, suitable RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Of course, it should be recognized that numerous other or additional collection modalities are also deemed appropriate, and that the cfRNA and/or ctDNA can be at least partially purified or temporarily adsorbed to a solid phase to so increase stability prior to further processing.

As will be readily appreciated, fractionation of plasma and extraction of ctDNA and cfRNA can be done in numerous manners. In one exemplary preferred aspect, whole blood in 10 mL tubes is centrifuged to fractionate plasma at 1600 rcf for 20 minutes. Appropriate centrifugation speeds can be calculated for various rotors following known conversions (e.g., RCF=1.1118×10⁻⁵×rpm², with r being rotor radius in cm). The so obtained plasma is then further centrifuged at 16,000 rcf for 10 minutes to remove cell debris. Of course, various alternative centrifugal protocols are also deemed suitable so long as the centrifugation will not lead to substantial cell lysis/maintains integrity of the blood cells (e.g., lysis of no more than 3%, or no more than 1%, or no more than 0.1%, or no more than 0.01%, or no more than 0.001% of all cells). cfDNA and cfRNA can then be extracted from a desirable volume (e.g., 2 mL) of plasma using Qiagen or other commercially available reagents. All isolated ctDNA and/or cfRNA are then kept in preferably bar-coded matrix storage tubes (e.g., with DNA stored at −4° C., RNA stored at −80° C., or reverse-transcribed to cDNA that is then stored at −4° C.).

Quantification of cfRNA can be performed in numerous manners, and contemplated methods include quantification by digital PCR methods, absolute quantification methods using external standards, and most typically relative quantification methods using internal standards (e.g., expressed as 2^{ΔΔCt}). For example, real-time qPCR amplification can be performed using an assay in a 10 μL reaction mix containing 2 μL cDNA, primers, and probe. β-actin can be used as an internal standard for the input level of ct-cDNA. A standard curve of samples with known concentrations of each analyte can be included in each PCR plate as well as positive and negative controls for each gene. Test samples are then identified by scanning the 2D barcode on the matrix tubes containing the nucleic acids. Delta Ct (dCT) were calculated from the Ct value derived from quantitative PCR (qPCR) amplification for each analyte subtracted by the Ct value of β-actin for each individual patient's blood sample. Relative expression of patient specimens is calculated using a standard curve of delta Cts of serial dilutions of Universal Human Reference RNA set at a gene expression value of 10 (when the delta CTs were plotted against the log concentration of each analyte). ctDNA can be analyzed in a similar fashion.

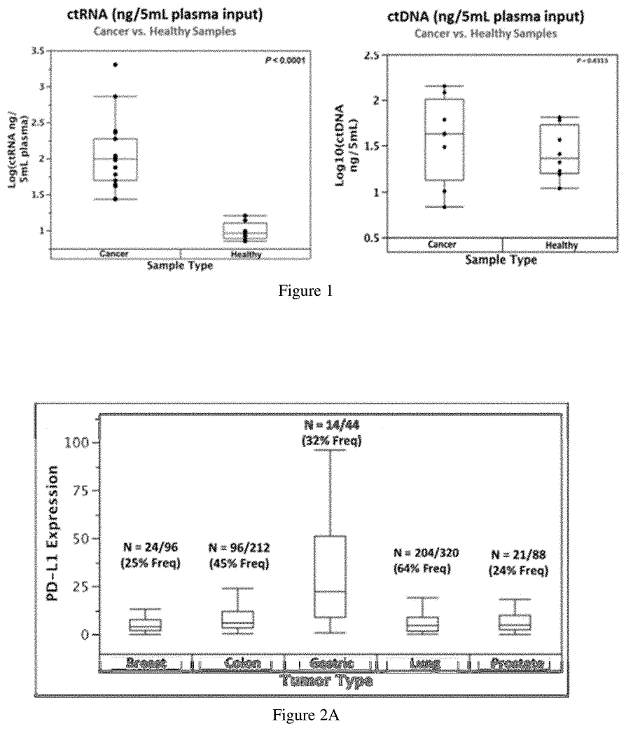

With regard to ctDNA, it should be noted that the accuracy of ctDNA in diagnostic tests has been in question since its adoption as a diagnostic tool for cancer. Issues with unusually high false positive rates must be addressed when relying on ctDNA in monitoring disease progression, but especially when considering the use of ctDNA in prediction of disease existence. As shown in FIG. 1, healthy individuals produce similar amounts of total ctDNA as cancer patients, however, levels of total cfRNA (e.g., as determined by quantitation using beta actin) are significantly low in healthy individuals. Moreover, and when cfRNA isolation protocols were performed under conditions that did not lead to substantial cell lysis, the levels of total cfRNA were significantly different between cancer patients and healthy individuals. Indeed, there was no overlap between the groups of healthy individuals thereby allowing the cancer patients to be distinguished by their total cfRNA levels. Conversely, there was overlap between the levels of ctDNA in cancer patients and healthy individuals. Therefore ctDNA could not distinguish between these two groups. In further contemplated methods, it should be appreciated that where total cfRNA is isolated, cfDNA may be removed and/or degraded using appropriate DNAses (e.g., using on-column digestion of DNA). Likewise, where ctDNA is isolated, cfRNA may be removed and/or degraded using appropriate RNAses. Moreover, the linear detection range for cfRNA (here: PD-L1) was significant when isolation protocols were performed under conditions that did not lead to substantial cell lysis as is shown in more detail below.

It should be noted that the term cfRNA includes full length RNA as well as fragments of full length RNA (which may have a length of 50-150 bases, 15-500 bases, or 500-1,000 bases, or more). Thus, cfRNA may represent a portion of an RNA, which may be between 100-80% of the full length RNA (typically mRNA), or between 80-60%, or between 60-40%, or between 40-20%, or even less. Moreover, it should be appreciated that the term cfRNA typically refers to a tumor-derived RNA (as opposed to an RNA from a non-tumor cell) and that the cfRNA may therefore be from a tumor cell of a solid tumor, a blood borne cancer, circulating tumor cells, and exosomes. Most typically, however, the cfRNA will be not be enclosed by a membrane (and as such be from a circulating tumor cell or exosome). Moreover, it should be appreciated that the cfRNA may be uniquely expressed in a tumor (e.g., as a function of drug resistance or in response to a treatment regimen, as a splice variant, etc.) or as a mutated form of a gene (e.g., as a fusion transcript, as a transcript of a gene having a single or multi-base mutation, etc.). Therefore, and viewed from a different perspective, contemplated cfRNA especially include transcripts that are unique to a tumor cell relative to a corresponding non-tumor cell, or significantly over-expressed (e.g., at least 3-fold, or at least 5-fold, or at least 10-fold) in a tumor cell relative to a corresponding non-tumor cell, or have a mutation (e.g., missense or nonsense mutation leading to a neoepitope) relative to a corresponding non-tumor cell.

Therefore, with respect to suitable target nucleic acids, it should be appreciated that appropriate targets particularly include genes that are relevant to a disease and/or treatment of a disease. For example, disease targets include one or more cancer associated genes, cancer specific genes, genes with patient and tumor-specific mutations (and especially those leading to the formation of neoepitopes), cancer driver genes, and genes known to be overexpressed in cancer. Consequently, suitable targets include those that encode ‘functional’ proteins (e.g., enzymes, receptors, transcription factors, etc.) and those that encode ‘non-functional’ proteins (e.g., structural proteins, tubulin, etc.). Viewed from a different perspective, suitable targets may also include targets that are specific to a diseased cell or organ (e.g., PCA3, PSA, for prostate, etc.), or targets that are more commonly found in different cancers, such as various mutations in KRAS (e.g., G12V, G12D, G12C, etc) or BRAF (e.g., V600E), etc. Exemplary targets validated by the inventors include AKT1, BRAF, CDK6, CYP3A4, ERBB3, FGFR1, JAK1, MAP2K1, AR-V7, ALK, BRCA1, CDKN2A, DDR2, ERBB4, FGFR2, JAK2, MET, AR, ARAF, BRCA2, CTNNB1, OPYD, FGF19, FGFR3, KOR, MTOR, PD-U, ATM, CCND1, CYP2C19, EGFR, FGF3, FLT3, KIT, NRAS, PD-1, BIM, CDK4, CYP2D6, HER2, FGF4, HRAS, KRAS, NRG1, TIM3, NTRK1, PTCH1, SMO, NTRK2, PTEN, STK11, NTRK3, RAF1, LAG3, TP53, PDGFRA, RET, TSC1, PIK3CA, RO-S1, TSC2, and UGT1A1.

Consequently, it should be appreciated that suitable treatment targets include one or more markers that are indicative of susceptibility of a diseased cell to treatment with a specific drug that targets a specific molecular entity. For example systems and methods presented herein may be useful to identify the presence and expression level of a specific kinase that is targeted by a kinase inhibitor, or the presence and expression level of a specific signaling receptor targeted by synthetic ligand, or the presence and expression level of a specific checkpoint receptor targeted by synthetic antagonist or antibody, etc., and suitable targets may also be grouped by indication as shown in Table 1 below.

TABLE 1

| | EGFR | ROS1 | KRAS | ALK | PD-L1 | NRAS | BRAF | AR-V7 |
|---|---|---|---|---|---|---|---|---|
| Lung | ✓ | ✓ | ✓ | ✓ | ✓ | | | |
| Colon | | | ✓ | | ✓ | ✓ | ✓ | |
| Prostate | | | | | ✓ | | | ✓ |
| Melanoma | | | | | ✓ | | ✓ | |

In addition to known markers such as tumor associated antigens and tumor specific antigens, it should also be appreciated that prior omics analysis of a patient's tumor may reveal the presence of one or more neoepitopes. For example, prior analysis can be done by tumor versus matched normal comparison of the whole genome or exome, preferably using incremental synchronous alignment as described in U.S. Pat. No. 9,721,062, and/or using RNAseq. In addition, proteomics analysis can be performed, most preferably using quantitative mass spectroscopic methods. Therefore, it should be appreciated that cfRNA may also be used to detect in a patient and tumor specific manner tumor RNA where the cfRNA contains such patient and tumor specific mutation (e.g., neoepitope). For example, such detection may be useful in monitoring treatment effect, particularly where the treatment is an immune therapy that targets the patient and tumor specific mutation (e.g., neoepitope). In another example, detection of a patient and tumor specific mutation may also reveal a (newly arisen) treatment target that may be treated with immune or chemotherapy.

Therefore, it should be appreciated that contemplated compositions and methods can be used in the discovery of disease associated markers, and more typically in quantification of suitable targets to so obtain information about presence of a mechanistic target for treatment and/or to obtain a quantitative proxy baseline for a cancer cell population to follow treatment or predict response development. For example, contemplated compositions and methods are especially suitable for immune therapy where the target is a neoepitope as expression and quantity of the neoepitope can be used to validate the neoepitope as a therapeutic target and to use the expression and quantity of the neoepitope as a proxy marker for treatment progress. Thus, it should be noted that cfRNA can be used to ascertain presence of expressed neoepitope before, during, and after treatment and as such allows to predict and/or quantitate treatment efficacy on an individual basis.

Alternatively, and among other preferred uses, cfRNA may be quantified to identify patients suitable for treatment with checkpoint inhibitors (e.g., targeting PD-1 and PD-L1). Such is especially useful as there is currently no convenient and non-invasive way to ascertain levels of PD-1 and PD-L1, which will inform a clinician if a patient will benefit from treatment with checkpoint inhibitors (e.g., nivolumab, pembrolizumab, atezolizumab, etc.). Indeed, immune checkpoints, such as programmed death ligand 1 (PD-L1) or its receptor, programmed death 1 (PD-1), appear to be Achilles' heels for multiple tumor types. PD-L1 not only provides immune escape for tumor cells but also turns on the apoptosis switch on activated T cells. Therapies that block this interaction have demonstrated promising clinical activity in several tumor types. Tumoral PD-L1 expression status has been shown to be prognostic in multiple tumor types, including melanoma (MEL), renal cell carcinoma (RCC), and non-small-cell lung cancer (NSCLC). In addition, tumoral PD-L1 expression appears to correlate closely with response to anti-PD-1 antibodies. However, no test is uniformly accepted as the standard for quantitating PD-L1 expression. Moreover, a few anti-PD-L1 antibodies are in clinical trial stages and two were already approved by FDA for treating NSCLC. Thus it is important to measure PD-L1 expression before giving the patient anti-PD-L1 immunotherapy. The inventors have now discovered that that PD-L1 expression and other immune therapy relevant cancer markers can be quantitated using cfRNA by analyzing the frequency and level of PD-L1 (and other marker) expression in cfRNA isolated from various cancer types as is shown in more detail below.

### EXAMPLES

Isolation of crRNA from whole blood: Whole blood was obtained by venipuncture and 10 ml were collected into cell-free RNA BCT® tubes or cell-free DNA BCT® tubes (Streck Inc.,7002 S. 109^{th} St., La Vista Nebr. 68128) containing RNA or DNA stabilizers, respectively. The sample tubes were then centrifuged at 1,600 rcf for 20 minutes, plasma was withdrawn and further centrifuged at 16,000 rcf for 10 minutes to remove cell debris. Plasma was used to isolate cfRNA using commercially available RNA isolation kits following the manufacturer's protocol with slight modification. Specifically, DNA was removed from the sample in an on-column DNAse digest.

In an alternative approach, cfRNA was also obtained in an automated manner using a robotic extraction method on QiaSymphony instrumentation (Qiagen, 19300 Germantown Road; Germantown, Md. 20874), slightly modified to accommodate for DNA removal where desired. The robotic extraction maintained approximately 12% DNA contamination in the cfRNA sample. We measured the relative expression of Excision Repair Cross-Complementing enzyme (ERCC1) vs beta actin in the same twenty-one NSCLC samples to determine whether there was a significant difference between the two extraction procedures. Notably, there was no statistical difference in the relative expression generated by the automated process and the manual process as shown in the table below. p=0.4111 (paired t-test; a statistically difference would have been p<0.05 for this test).

| | | | | Manual | | | | |
|---|---|---|---|---|---|---|---|---|
| | | | | Relative | | | | QIAsymphony |
| | Manual | Manual | Manual | ERCC1 | QIAsymphony | QIAsymphony | QIAsymphony | Relative ERCC1 |
| Sample # | ERCC1 CTs | ACTB CTs | 2\hat{ }-dCTs | Expression | ERCC1 CTs | ACTB CTs | 2\hat{ }-dCTs | Expression |
| 1 | 30.09 | 22.31 | 0.00 | 2.75 | 29.76 | 22.27 | 0.01 | 3.37 |
| 2 | 31.22 | 23.46 | 0.00 | 2.79 | 31.30 | 23.35 | 0.00 | 2.47 |
| 3 | 31.50 | 23.65 | 0.00 | 2.64 | 30.64 | 23.48 | 0.01 | 4.26 |
| 4 | 32.02 | 24.35 | 0.00 | 2.97 | 30.58 | 23.41 | 0.01 | 4.21 |
| 5 | 31.60 | 25.12 | 0.01 | 6.78 | 31.79 | 24.20 | 0.01 | 3.15 |
| 6 | 30.30 | 23.54 | 0.01 | 5.61 | 30.47 | 22.96 | 0.01 | 3.31 |
| 7 | 30.94 | 22.33 | 0.00 | 1.54 | 30.49 | 20.35 | 0.00 | 0.54 |
| 8 | 31.96 | 24.60 | 0.01 | 3.67 | 31.16 | 24.05 | 0.01 | 4.38 |
| 9 | 31.74 | 23.29 | 0.00 | 1.74 | 30.67 | 23.52 | 0.01 | 4.26 |
| 10 | 31.73 | 24.64 | 0.01 | 4.43 | 31.87 | 24.18 | 0.00 | 2.94 |
| 11 | 29.77 | 22.23 | 0.01 | 3.25 | 30.52 | 22.21 | 0.00 | 1.92 |
| 12 | 31.44 | 24.19 | 0.01 | 3.99 | 30.85 | 24.20 | 0.01 | 6.07 |
| 13 | 31.48 | 23.73 | 0.00 | 2.82 | 31.28 | 23.59 | 0.00 | 2.94 |
| 14 | 29.61 | 21.91 | 0.00 | 2.92 | 29.11 | 21.58 | 0.01 | 3.28 |
| 15 | 30.47 | 24.20 | 0.01 | 6.76 | 30.35 | 23.45 | 0.01 | 6.59 |
| 16 | 30.30 | 23.91 | 0.01 | 6.21 | 31.56 | 24.31 | 0.01 | 5.19 |
| 17 | 30.55 | 23.73 | 0.01 | 4.60 | 29.60 | 22.16 | 0.01 | 4.53 |
| 18 | 30.77 | 23.92 | 0.01 | 4.49 | 32.34 | 23.87 | 0.00 | 2.23 |
| 19 | 30.90 | 24.79 | 0.01 | 7.56 | 32.17 | 24.21 | 0.00 | 3.16 |
| 20 | 31.90 | 23.52 | 0.00 | 1.57 | 31.66 | 23.24 | 0.00 | 2.31 |
| 21 | 30.42 | 23.85 | 0.01 | 5.48 | 30.50 | 23.29 | 0.01 | 5.33 |

Custom kit from Qiagen (QiaSymphony Circulating NA kit #1074536) included two virus extraction kits in one custom kit (the virus kits are called QiaSymphony DSP Virus/Pathogen Midi Kit Version 1 #937055). Analyses were run within single, proprietary program on Qiagen instrument (custom program protocol CF 2000S_CR21040_ID993; from Qiagen).

Quantification of cfRNA: Unless otherwise noted, quantification was performed using relative quantification via rtPCT and gene specific primer pairs along with primer pairs for beta-actin as internal control. For example, amplifications were performed using an assay in a 10 μL reaction mix containing 2 μL cDNA, primers, and probe. β-actin can be used as an internal standard for the input level of ct-cDNA. A standard curve of samples with known concentrations of each analyte wad included in each PCR plate as well as positive and negative controls for each gene. Test samples were identified by scanning the 2D barcode on the matrix tubes containing the nucleic acids. Delta Ct (dCT) were calculated from the Ct value derived from quantitative PCR (qPCR) amplification for each analyte subtracted by the Ct value of β-actin for each individual patient's blood sample. Relative expression of patient specimens was calculated using a standard curve of delta Cts of serial dilutions of Universal Human Reference RNA set at a gene expression value of 10 (when the delta CTs were plotted against the log concentration of each analyte). ctDNA was analyzed in a similar fashion.

Delta Cts vs. log₁₀ Relative Gene Expression (standard curves) for each gene test were captured over hundreds of PCR plates of reactions (historical reactions). A linear regression analysis was performed for each assay and used to calculate gene expression from a single point from the original standard curve going forward.

Notably, as is shown in FIG. 1, where ctDNA was quantified from healthy donors and cancer patients, non-small cancer (NSCLC), 10 cancer and 9 healthy individuals. No statistically significant difference could be overserved with total ctDNA between the two populations. In contrast, total cfRNA quantities (as measured by β-actin) were significant different between the two populations, indicating that measurement of total cfRNA may be a valid indicator for the presence of cancer.

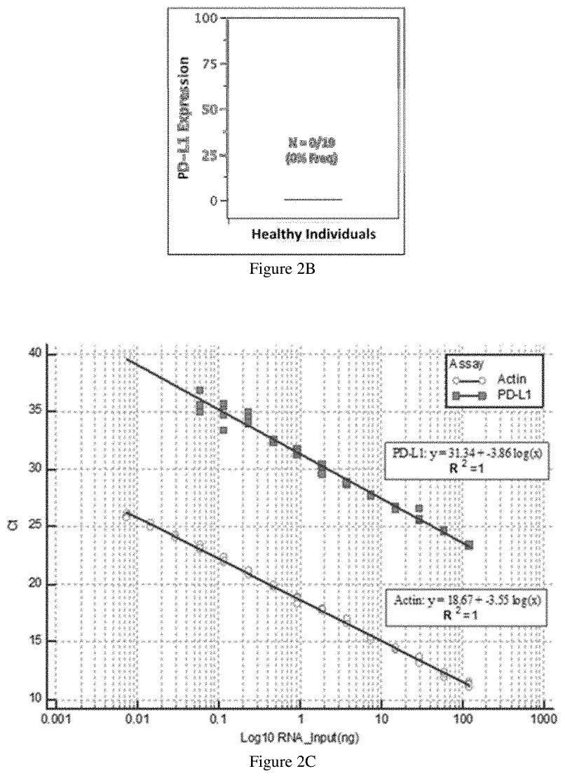

The inventors then investigated whether the above results could be confirmed across various other cancer types and selected genes (e.g., PD-L1) and analyzed blood samples from selected patients diagnosed with breast cancer, colon cancer, gastric cancer, lung cancer, and prostate cancer. In this series of tests, relative expression of PD-L1cfRNA was quantitated, and the results are depicted in FIG. 2A. Interestingly, not all cancers expressed PD-L1 as shown in FIG. 2A, and the frequencies of positivity in the various cancers was concordant with the published expression of PD-L1 using IHC in solid tissue. PD-L1cfRNA was not detectable in healthy patients as can be seen from FIG. 2B.

Assay Validation—Accuracy: Accuracy of an exemplary PD-L1 Expression Assay was determined by comparing the results generated by the present PD-L1 assay (“LiquidGeneDx”) from 61 clinical samples against a digital PCR PD-L1 assay (lab developed reference method, an alternative PD-L1 detection assay). The results were used to determine the clinical sensitivity and clinical specificity of the assay. The accuracy results from the present PD-L1 assay and the digital PCR PD-L1 assay are summarized in Table 2.

TABLE 2

| | Positive Agreement | Negative Agreement |
|---|---|---|
| | (LiquidGeneDx vs Digital PCR) | (LiquidGeneDx vs Digital PCR) |
| PD- | 91% | 94% |
| L1 | | |

Assay Validation—Limit of Detection (LOD): Analytical sensitivity of the present PD-L1 assay (“LiquidGeneDx”) was determined by 20 replicates at a 95% detection rate. cfRNA was extracted from patients' plasma, reverse-transcribed using random hexamers to cDNA and pre-amplified using Thermo Fisher's pre-amplification product Taqman® Preamp Master Mix with PD-L1 and beta-actin primers for 10 cycles per the manufacturer's instructions. The resulting pre-amplified cDNA was diluted in 2-fold increments with cDNA from patients' plasma negative for PD-L1. All dilution samples were examined by LiquidGeneDx for the minimum amount of PD-L1 cDNA required for amplification and successful PCR. Then 20 replicates at the presumptive LOD level were used to confirm the final LOD. The limit of detection (LOD) acceptance criteria in this study was determined as the lowest concentration at which all 20 replicates generated a 95% above the detection rate. If 20 replicates could not generate a 95% above detection rate, the next higher concentration of dilution samples were used as presumptive LOD to repeat with 20 replicates. A summary of LOD study results is shown in Table 3 in which the * denotes the final LOD.

TABLE 3

| | Valid Positive Results/Total Tested | | | | | |
|---|---|---|---|---|---|---|
| PD-L1 Dilution Sample | 1.884 ng | 0.941 ng | 0.471 ng | 0.236 ng | 0.118 ng* | 0.059 ng |
| PD-L1 Expression | 4/4 | 4/4 | 4/4 | 4/4 | 20/20 | 15/20 |

Assay Validation—Limit of Detection (LOD): The precision panel included a low positive PD-L1 sample, a medium positive PD-L1 sample, a high negative PD-L1 sample, positive control, and no-template control. All positive samples were made from a PD-L1 positive cancer cell line. Each precision panel was examined in quadruplicate per run, 2 runs per instrument for 2 instruments per day for total of 3 days (consecutive or non-consecutive) by three different operators (Op). Each sample of the precision panel generated total 48 data points across 3 days. The study design is illustrated in Table 4.

TABLE 4

| | |
|---|---|
| Instrument 1 | Instrument 2 |

| | | | | |
|---|---|---|---|---|
| Day 1 | Op 1 | Op 2 | Op 3 | Op 1 |
| Day 2 | Op 2 | Op 3 | Op 1 | Op 2 |
| Day 3 | Op 3 | Op 1 | Op 2 | Op 3 |

The intra-assay precision was done using two instruments, two operators, one day, and four replicates per samples. Result concordance for all replicates are 96% or above. Table 5 is an exemplary summary of the intra-assay precision.

TABLE 5

| | | | Operator 1 | | | Operator 2 | | |
|---|---|---|---|---|---|---|---|---|
| Sample | | Expression | Run 1 | Run 2 | Run 3 | Run 1 | Run 2 | Run 3 |
| 1 | Positive | PD-L1 | 100% | 96% | 100% | 100% | 100% | 100% |
| 2 | Negative | Water | 100% | 100% | 100% | 100% | 100% | 100% |

Two instruments, two operators, three runs were done over three days, and quadruplicate runs were tested for inter-assay precision. Result concordance reached 96% or above for all replicates across independent runs. Result summary is listed in Table 6.

TABLE 6

| Comparison | Standard | Result |
|---|---|---|
| Operator #1 vs. Operator #2 | Result Agreement | 99% |
| Operator #1, between runs | Result Agreement | 96% |
| Operator #2, between runs | Result Agreement | 100% |

Assay Validation—Linear Range: Quantitative linear range of the present PD-L1 assay (“LiquidGeneDx”) was determined by diluting PD-L1-positive patients' cDNA from cfRNA into a pooled negative matrix (PD-L1-negative cDNA from cfRNA). ct RNA was extracted from patients' plasma, reverse-transcribed using random hexamers to cDNA and pre-amplified using Thermo Fisher's pre-amplification product Taqman® Preamp Master Mix with PD-L1 and beta-actin primers for 10 cycles per the manufacturer's instructions. The resulting pre-amplified cDNA was diluted in 2-fold increments with cDNA from patients' plasma negative for PD-L1. All dilution samples were examined by LiquidGeneDx PD-L1 to determine its quantitative linear range. FIG. 2C shows the final linear range. The linear portion of the line extends to a Ct of approximately 32.5. Beta-actin and PD-L1 slopes are also concordant.

Assay Validation—Specificity: Test samples were prepared by serial dilution of human PD-L1 cell line cDNA in TE buffer matrix. Concentration of target analyte for medium positive samples was 4 times the LOD concentration. Medium-positive samples with each interferent (one analyte with each interferent) as well as baseline samples were examined in triplicate by the present PD-L1 assay (“LiquidGeneDx”). Table 7 is the list of interferents and their testing concentration. All samples with testing concentration of different interferents were still determined as positive by the LiquidGeneDx PD-L1 assay.

TABLE 7

| Interferents | Interference Concentration |
|---|---|
| Buffer ACL | 0.1% in total volume |
| Buffer ACB | 0.2% in total volume |
| Buffer ACW1 | 1% in total volume |
| Buffer ACW2 | 1% in total volume |
| Buffer AVE | 1% in total volume |
| Albumin | 2 mg/mL |
| Casein | 2 mg/mL |
| Hemoglobin | 0.4 mg/mL |
| Actin DNA/RNA mix | 1 ng in total |

Notably, all samples with testing concentration of different interferents were still determined as positive by the LiquidGeneDx PD-L1 assay.

The present PD-L1 assay (“LiquidGeneDx”) was designed as a real-time PCR assay to detect expression of the PD-L1 gene and other genes in blood of cancers patients. Among other benefits, such measurements can inform a clinician about the likely treatment success with a specific drug (e.g., anti-PD-1 antibody) before and during drug therapy.

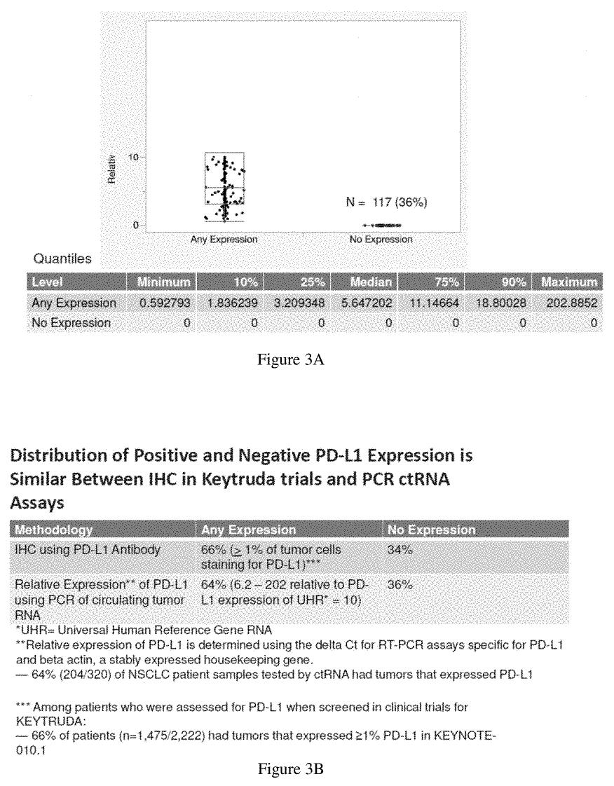

Based on the above findings that cfRNA can be accurately quantified, the inventors sought to determine whether the quantified cfRNA levels would also correlate with known analyte levels measured by conventional methods such as FISH, mass spectroscopy, etc. More specifically, the frequency and strength of PD-L1 expression was measured by cfRNA from the plasma of 320 consecutive NSCLC patients using LiquidGenomicsDx and compared to the frequency of positive patients in the Keynote Trial, a registration trial of pembrolizumab (Keytruda), using a tissue IHC test. Notably, 66% of NSCLC patients (1,475/2,222) in the Keynote trial had any expression of PD-L1 by IHC (>1% of cells positive), while 64% of NSCLC (204/320) patients with blood-based cfRNA testing of PD-L1 were positive as can be seen from FIGS. 3A and 3B. Remarkably, there was no significant difference in PD-L1 status between the two analytical methods, but the cfRNA testing afforded quantitative data.

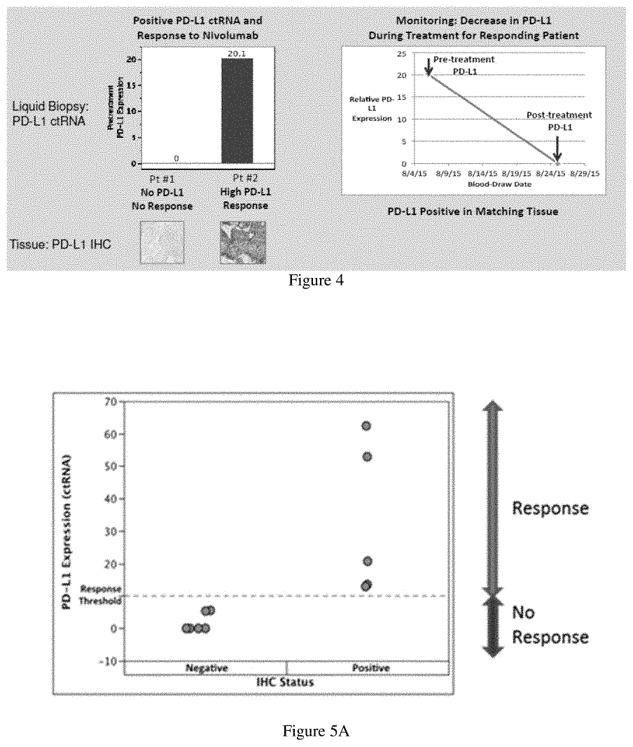

Notably, the difference in PD-L1 status (i.e., PD-L1 positive or PD-L1 negative) of two selected patients (Pt#1 and Pt#2) also correlated well with IHC analysis and treatment response with nivolumab as can be seen from FIG. 4. Here, two squamous cell lung cancer patients were treated with the anti-PD-1 antibody nivolumab. Patient 1 had no expression of PD-L1 in the tissue or in the blood using cfRNA measurement. Patient 1 did not respond to nivolumab. Tumor growth was documented by CT scan and the patient expired rapidly. In contrast, Patient 2 had high levels of PD-L1 in the tissue and in the blood at baseline using cfRNA measurement. Patient 2 responded to nivolumab with a durable response over several cycles of the drug. The response was documented by CT scan with dramatic tumor shrinkage. Interestingly, the high levels of gene expression in the blood of this patient (measured by cfRNA) disappeared after three and a half weeks while the patient continued to respond.

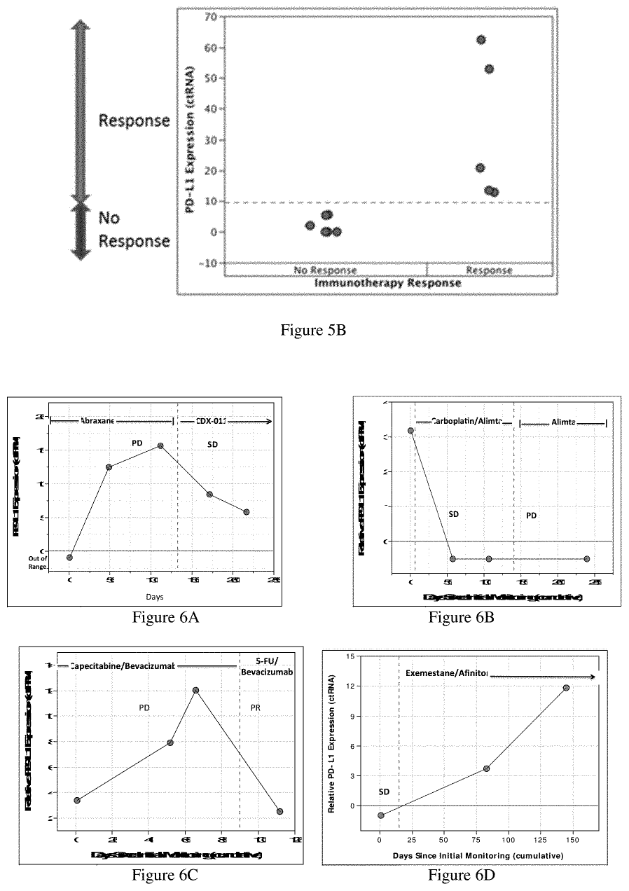

Based on the above observed correlation, the inventors set out to investigate whether or not expression levels of PD-L1 cfRNA could provide threshold levels suitable for response prediction to treatment with nivolumab or other therapeutics interfering with PD1/PD-L1 signaling. To that end, PD-L1 expression was measured in NSCLC patient plasma using cfRNA and compared with IHC status. FIG. 5A shows the correlation between treatment response status with an anti-PD-L1 therapeutic and PD-L1 status as determined by IHC and PD-L1 expression above response threshold by cfRNA. Patients determined to be treatment responders were also determined by IHC as PD-L1 positive, while all patients determined to be non-responders to treatment were determined by IHC as PD-L1 negative. Remarkably, the same separation between responders and non-responders could be achieved using PD-L1 cfRNA levels when a response threshold was applied to then data. In this example, a relative expression threshold of 10 accurately separated responders from non-responders. FIG. 5B shows that a cfRNA response threshold of ΔΔCT>10 for PD-L1 relative to β-actin predicts positive response to a PD1/PD-L1 checkpoint inhibitor (here: nivolumab). All responders to nivolumab expressed PD-L1 above the threshold level prior to treatment.

The inventors further investigated if PD-L1 cfRNA expression levels could be used in other cancer treatments as an indicator for progressive disease (PD), stable disease (SD), and/or partial response (PR). To that end, dynamic changes in PD-L1 measured by cfRNA were found during the course of therapy under various treatment regimens as is exemplarily shown in FIGS. 6A-6D. Panel A shows the relative expression levels for PD-L1 over the course of treatment of breast cancer with abraxane in a patient with progressive disease. The lack of response to treatment is reflected in the rise of PD-L1 cfRNA, and abraxane treatment was discontinued in favor of treatment with CDX-011 (glembatumumab vedotin). As can be seen from FIG. 6A, treatment with CDX-011 lead to disease stabilization, which is also reflected in a decrease of PD-L1 cfRNA. Similarly, as can be taken from FIG. 6B, a lung cancer patient was treated at stable disease with a carboplatin/alimta combination therapy, and initially high levels of PD-L1 cfRNA dramatically decreased as the patient showed partial response. In the case of colon cancer, a patient with progressive disease was treated with capecitabine and bevacizumab. During treatment, relative PD-L1 cfRNA expression significantly increased. Upon treatment of the cancer with 5-FU and bevacizumab, the patient had a partial response with concomitant significant drop in PD-L1 cfRNA levels as can be taken from FIG. 6C. Therefore, the inventors contemplate that quantitative levels of PD-L1 cfRNA can also accurately serve to monitor treatment response.

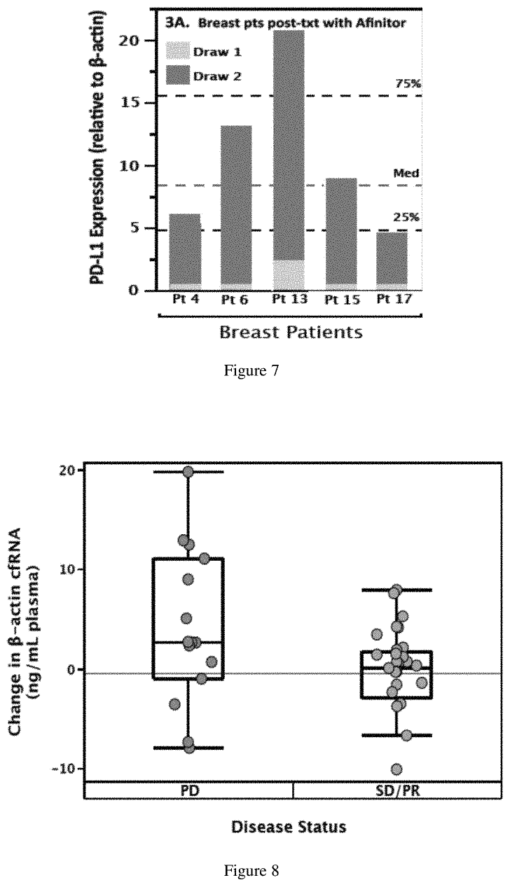

In yet another example, the inventors observed a rapid increase in PD-L1 cfRNA in a patient with stable disease breast cancer upon treatment with exemestane/afinitor as is shown in FIG. 6D. Notably, the patient did not have measureable quantities of PD-L1 cfRNA before treatment. Based on this observation, the inventors tested further breast cancer patient samples that underwent afinitor treatment and exemplary results are depicted in FIG. 7. As is readily apparent, relative PD-L1 cfRNA significantly increased post treatment at the second blood draw to levels suitable for treatment with PD1/PD-L1 checkpoint inhibitors. Therefore, the inventors also contemplate that cancer treatments (especially those using drugs other than PD1/PD-L1 checkpoint inhibitors) can be followed by at least monitoring PD-L1 cfRNA to identify emergence of PD-L1 cfRNA expression, which can then serve as an indicator of treatment with a PD1/PD-L1 checkpoint inhibitors. Viewed from a different perspective, detection and quantitation of previously not detectable PD-L1 cfRNA expression during a cancer treatment may be used as an indicator to (additionally) treat a patient with a PD1/PD-L1 checkpoint inhibitor.

Interestingly, disease status of cancer also paralleled to at least some extent β-actin cfRNA as can be seen from FIG. 8. Blood was drawn from patients under various therapies every 6-8 weeks, at the same time that the CT scans were done. cfRNA was extracted from plasma of 45 patients with metastatic breast cancer, and 30 patients completed the first two cycles of therapy: 6/6 patients with PR showed either no change (NC) or a decrease (DEC) in levels of β-actin cfRNA, 13/16 patients with SD showed NC or DEC in cfRNA levels, and 6/8 patients with PD underwent increases (INC) in levels of cfRNA. CfRNA was reverse transcribed with random hexamers to cDNA. Levels of cfRNA were quantitated by RT-qPCR and correlated with patient response (PR/SD/PD), as determined by CT scans. Levels of gene expression in cfRNA (including PD-L1 and HER2) were monitored in patients across blood draws. Notably, β-actin cfRNA levels of breast cancer patients with progressive disease was higher than β-actin cfRNA levels of patients with stable disease and/or partial response. Thus, it should be appreciated that an increase in β-actin cfRNA levels can serve as a leading indicator of disease status, and especially of progressive disease in patients already diagnosed with cancer.

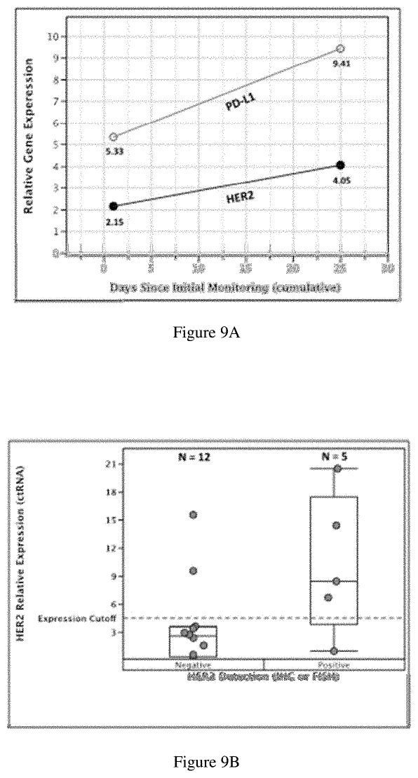

Upon further investigation of breast cancer samples, the inventors also discovered that HER2 cfRNA in tumors appeared to be co-expressed or co-regulated with PD-L1 as is shown in FIG. 9A. On this basis, the inventors then used HER2 status classification by immune histochemical analysis using antiHER2 antibodies (IHC) to correlate IHC-HER2 status with quantitative relative expression of HER2 as measured by cfRNA levels. Remarkably, there was a significant correlation (82% concordance) between HER2 cfRNA expression levels and IHC HER2 status where a ΔΔCT>5 for HER2 relative to β-actin was applied as is exemplarily shown in FIG. 9B. Therefore, it is contemplated that HER2 status may also be determined using detection and quantification of HER2 cfRNA using an expression threshold as provided above.

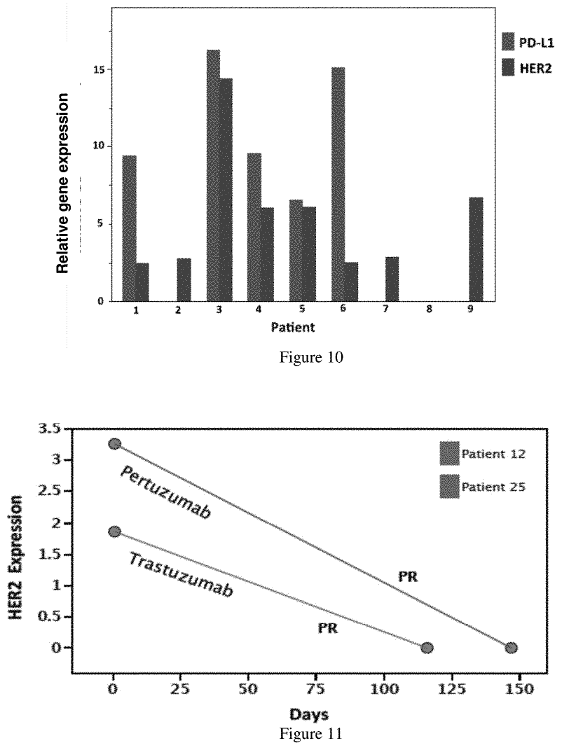

In further experiments, the inventors also discovered that that HER2 cfRNA in at least some gastric tumors also appeared to be co-expressed or co-regulated with PD-L1 as is shown in FIG. 10. Such finding is particularly notable as it is known that about 15% of all gastric cancers do express HER2. Consequently, the inventors contemplate methods of detecting or quantifying HER2 cfRNA in patients with gastric cancer. Furthermore, the inventors also contemplate that one or more immune checkpoint genes (e.g., PD-L1, TIM3, LAG3) as measured by cfRNA may be used as proxy markers for other cancer specific markers or tumor associated markers (e.g., CEA, PSA, MUC1, brachyury, etc.).

As will be readily appreciated, the quantification of HER2 cfRNA levels may also be employed to follow treatment, and particularly to assess whether or not treatment with an anti-HER2 drug has therapeutic effect. For example, partial treatment response to two anti-HER2 drugs (pertuzumab and trustuzumab) in two exemplary patients (patients 25 and 12, respectively) of a cohort of metastatic breast cancer patients showed that positive response directly correlated with a reduction of cfRNA as is depicted in FIG. 11. Indeed, past three months of treatment no detectable quantities of HER2 cfRNA were present.

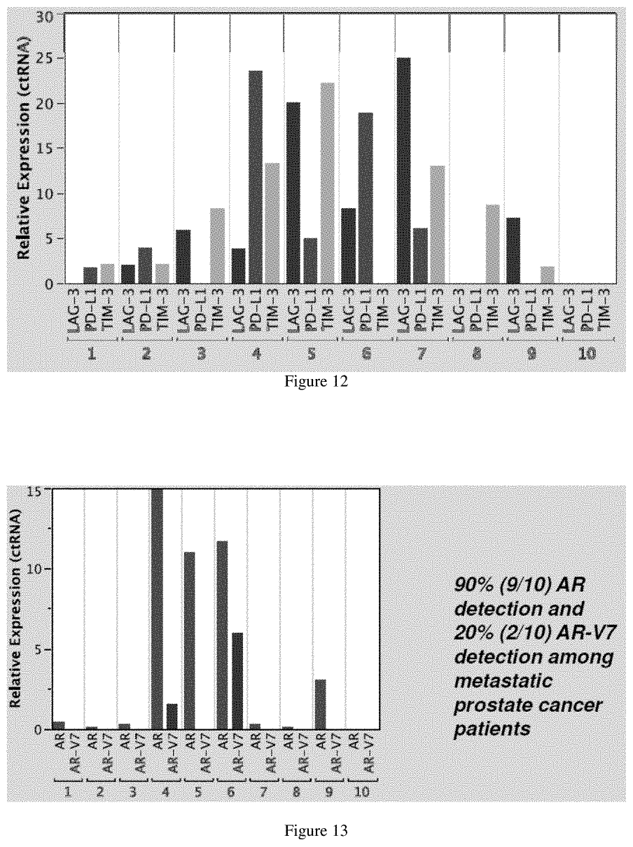

Based on the observed co-expression or co-regulation, the inventors then investigated whether or not other cfRNA levels for immune checkpoint related genes would correlate with PD-L1 cfRNA levels and exemplary results are depicted in FIG. 12. Here, cfRNA levels for PD-L1, TIM3, and LAG3 were measured from blood samples of prostate cancer patients. Notably, in all but one sample more than one checkpoint related gene was strongly expressed. Interestingly and importantly, levels of TIM3 and LAG3, the former of which has been shown to serve as an escape mechanism or resistance factor for PD-1 or PD-L1 inhibition, often mirrored PD-L1 expression, underscoring a need to address all checkpoint proteins besides PD-1 and PD-L1. Therefore, it should be appreciated that cfRNA levels for immune checkpoint relevant genes may be analyzed for cancer patients to so obtain an immune signature or the patient, and the appropriate treatment with more than one checkpoint inhibition drug may be then be advised. As will be appreciated, suitable threshold values for the genes can be established following the methods described for PD-L1 and HER2 above.

In still further aspects of the inventive subject matter, various alternate cfRNA species were demonstrated to quantitatively distinguish healthy individuals from those afflicted with cancer and/or to predict treatment response. For example, the detection of the splice variant 7 of the androgen receptor (AR-V7) has been an important consideration for the treatment of prostate cancer with hormone therapy. The inventors therefore investigated whether or not hormone therapy resistance is associated with prostate cancer tumor growth and detection of AR-V7 via detection and quantification of AR-V7 cfRNA. FIG. 13 depicts exemplary results for AR and AR-V7 gene expression via cfRNA methods using plasma from prostate cancer patients. AR-V7 was also measured using IHC technology from CTCs from the same patients. Notably, the results from CTCs and cfRNA for AR-V7 were concordant (data not shown).

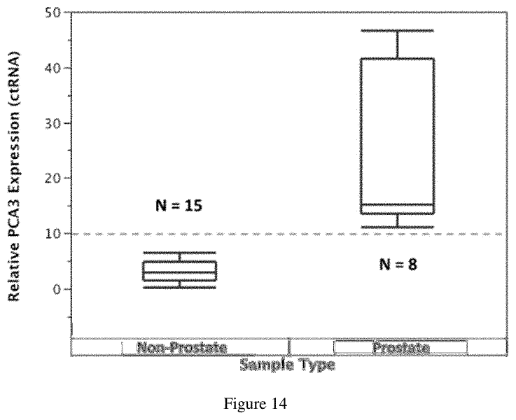

Furthermore, PCA3 was identified as a marker for prostate cancer in a test in which PCA3 cfRNA was detected and quantified in plasma from prostate cancer patients and in which non-prostate cancer patient samples had relatively low to non-detectable levels. Non-prostate cancer patients were NSCLC and CRC patients. As can be taken from FIG. 14, PCA3 was shown to be differentially expressed between the two groups (non-overlapping medians between prostate and non-prostate cancer patients) by cfRNA, indicating that the non-invasive blood based cfRNA test may be used to detect prostate cancer. Once more, based on a priori knowledge of the tested population, a threshold value (here: ΔΔCT>10 for PCA3 relative to β-actin) for expression could be established as is exemplarily depicted in FIG. 14.

In yet a further study, the inventors used analysis of total cell-free circulating tumor RNA (cfRNA) extracted from plasma of cancer patients (pts) as a tool to measure dynamic changes in gene expression as well as in total levels of nucleic acids including cfRNA. These analyses provided yet again insight into disease status and allowed predicting outcome to anti-tumoral therapy.

More specifically, blood was drawn from pts under various treatments (tx) every 6-8 weeks, at the same time that CT scans were done. CfRNA was extracted from the resulting plasma and reverse transcribed with random hexamers to cDNA as described above. Levels of total cfRNA were quantitated by RT-qPCR and correlated with pt response (PR/SD/PD), as determined by CT scans. In this study, a total of 30 lung cancer pts were enrolled in a 2-year clinical study. Ethnicities included: 73% (22/30) Caucasian, 20% (6/30) Hispanic, and 7% (2/30) other. Non-SQCC were 87% (26/30) of the total. 23 pts completed the first two cycles of tx. Of these, 6/8 pts with progressive disease (PD) showed increased (INC) levels of total cfRNA, 8/12 pts with stable disease (SD) showed either no change (NC) or decreased (DEC) total cfRNA, and 3/3 pts with partial response (PR) had DEC total cfRNA, corresponding to 74% concordance between total cfRNA and pt response. PD-L1 expression measured in plasma cfRNA matched that of tissue in 7/10 pts. In the one pt where PD-L1 was negative in blood and positive in tissue, the pt progressed on pembrolizumab. Among 7 pts treated with immunotherapy (nivolumab, pembrolizumab, atezolizumab), 3/3 pts with PD showed INC PD-L1 cfRNA expression, 3/3 pts with SD had NC in PD-L1 cfRNA, and 1 pt with PR showed DEC PD-L1 cfRNA, corresponding to 100% correlation between PD-L1 expression levels and pt response. Upon treatment, a significant concordance was observed between clinical response and changes in plasma cfRNA levels in NSCLC pts (74%). Detection of PD-L1 expression in pt plasma also correlated with results obtained from tissue of same pts (70%). While on targeted therapy, levels of PD-L1 expression correlated with response in 7/7 pts. It can therefore be concluded that cfRNA levels can indicate tx response, and PD-L1 in plasma could be used to monitor response to immunotherapy.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of predicting treatment response of an individual with cancer to treatment with a checkpoint inhibitor, comprising:
obtaining blood from the individual and isolating cfRNA from the blood, wherein the cfRNA encodes a checkpoint inhibition gene;
quantifying the cfRNA using quantitative PCR method; and
predicting a positive treatment response when the quantity of the cfRNA is above a threshold level.

2. The method of claim 1 wherein the checkpoint inhibitor is an antibody against PD1 or PD-L1 and wherein the cfRNA is PD-L1 cfRNA.

3. The method of claim 1 wherein the step of isolating the cfRNA uses RNA stabilization and cell preservation.

4. The method of claim 1 wherein the quantitative PCR method includes real time PCR.

5. The method of claim 1 wherein the step of quantifying uses an β-actin as an internal standard.

6. The method of claim 1 wherein the threshold level is ΔΔCT>10 for PD-L1 relative to β-actin.

7. The method of claim 1 further comprising a step of quantifying at least a second cfRNA using the quantitative PCR method.

8. The method of claim 7 wherein the at least a second cfRNA encodes TIM3 or LAG3.

9. The method of claim 1 further comprising a step of quantifying at least a second cfRNA, wherein the at least second cfRNA encodes a gene having a tumor and patient specific mutation, a tumor associated gene, or a cancer specific gene.

10. A method of monitoring treatment of an individual with cancer, comprising:
obtaining blood from the individual and isolating cfRNA from the blood, wherein the cfRNA encodes a checkpoint inhibition gene, or wherein the cfRNA encodes a tumor associated or cancer specific gene, or wherein the cfRNA encodes a gene having a tumor and patient specific mutation;
quantifying the cfRNA using quantitative PCR method; and
updating a patient record using the quantity of the cfRNA.

11. The method of claim 10 wherein the checkpoint inhibition gene is PD-L1, TIM3, or LAG3.

12. The method of claim 10 wherein the tumor associated or cancer specific gene is CEA, MUC1, brachyury, HER2, PCA3, or AR-V7.

13. The method of claim 10 wherein the gene having a tumor and patient specific mutation encodes a neoepitope.

14. The method of claim 10 wherein the step of isolating the cfRNA uses RNA stabilization and cell preservation.

15. The method of claim 10 wherein the quantitative PCR method includes real time PCR.

16. The method of claim 10 wherein the step of quantifying uses β-actin as an internal standard.

17. The method of claim 10 wherein the patient record is updated when the quantity of the cfRNA is ΔΔCT>5 for HER2 relative to β-actin or ΔΔCT>10 for PCA3 relative to β-actin.

18. A method of detecting prostate cancer, comprising:
obtaining blood from the individual and isolating cfRNA from the blood, wherein the cfRNA encodes PCA3 or a splice variant 7 of an androgen receptor;
quantifying the cfRNA using quantitative PCR method; and
diagnosing the individual as having cancer when the cfRNA quantity is above a threshold level.

19. The method of claim 18 wherein the individual is diagnosed as having cancer when the quantity of PCA3 cfRNA is ΔΔCT>10 relative to β-actin.

20. The method of claim 18 further comprising a step of quantifying at least a second cfRNA, wherein the at least second cfRNA encodes a gene having a tumor and patient specific mutation, a tumor associated gene, a cancer specific gene, or a checkpoint inhibition gene.

21. The method of claim 20 wherein the second cfRNA encodes PD-L1, LAG3, TIM3, AR-V7, PSA, and PSMA.

22. A method of treating a cancer, comprising:
administering a drug to an individual diagnosed with a PD-L1 negative cancer;
monitoring treatment of the individual by isolating cfRNA from the blood, wherein the cfRNA encodes PD-L1;
quantifying the cfRNA using quantitative PCR method; and
including a checkpoint inhibitor to the treatment upon detection of the cfRNA.

23. The method of claim 22 wherein the PD-L1 negative cancer is a solid cancer.

24. The method of claim 23 wherein the solid cancer is breast cancer.

25. The method of claim 22 wherein the drug is afinitor.

26. The method of claim 22 wherein the step of quantifying cfRNA uses real-time PCR.

27. The method of claim 22 wherein the checkpoint inhibitor is included when the cfRNA is detected and increases over time.

28. The method of claim 22 wherein the checkpoint inhibitor is included when the cfRNA is detected and the cfRNA level is ΔΔCT>10 relative to β-actin.

29. A method of determining an immune signature in a patient, comprising:
determining quantities of distinct cfRNA molecules in blood of an individual, wherein the cfRNA molecules encode distinct checkpoint inhibition genes;
wherein the step of determining is performed prior to or during treatment with at least one of a checkpoint inhibitor, a chemotherapeutic drug, an immune therapeutic drug, and radiation treatment.

30. The method of claim 29 wherein at least one of the distinct cfRNA molecules encodes PD-L1, LAG3, or TIM3.

31. The method of claim 29 wherein the step of determining quantities comprises real time PCR.

32. Use of cfRNA to predict a treatment response of an individual with cancer to treatment with a checkpoint inhibitor, wherein the cfRNA encodes a checkpoint inhibition gene, and wherein the cfRNA is above a threshold level.

33. The use of claim 32 wherein the checkpoint inhibitor is an antibody against PD1 or PD-L1 and wherein the cfRNA is PD-L1 cfRNA.

34. The use of claim 32 wherein the threshold level is ΔΔCT>10 for PD-L1 relative to β-actin.

35. Use of cfRNA to monitor treatment of an individual with cancer, wherein the cfRNA encodes a checkpoint inhibition gene, or wherein the cfRNA encodes a tumor associated or cancer specific gene, or wherein the cfRNA encodes a gene having a tumor and patient specific mutation.

36. The use of claim 35 wherein the checkpoint inhibition gene is PD-L1, TIM3, or LAG3.

37. The use of claim 35 wherein the tumor associated or cancer specific gene is CEA, MUC1, brachyury, HER2, PCA3, or AR-V7.

38. The use of claim 35 wherein the gene having a tumor and patient specific mutation encodes a neoepitope.

39. Use of a cfRNA encoding PCA3 or a splice variant 7 of an androgen receptor in the detection of prostate cancer in an individual.

40. The use of claim 39 wherein the individual is diagnosed as having prostate cancer when the cfRNA quantity is above a threshold level.

41. The use of claim 40 wherein the threshold level for PCA3 cfRNA is ΔΔCT>10 relative to β-actin.
